(19) United States
(12) Patent Application Publication
(10) Pub. No.: US 2002/0069872 A1
(43) Pub. Date: Jun. 13, 2002

(54) Mouthpiece

(76) Inventors: Lewis George Gradon, Auckland (NZ); Nicholas Charles Alan Smith, Auckland (NZ); Alastair Edwin McAuley, Auckland (NZ); Mark Joseph Haycock, Auckland (NZ); Chris Earl Nightingale, Auckland (NZ)

Correspondence Address: Trexler, Bushnell, Giangiorgi,, Blackstone & Marr, Ltd., 36th Floor, 105 W. Adams, Chicago, IL, 60603, US

(21) Appl. No.: 09/961,743

(22) Filed: Sep. 24, 2001

(30) Foreign Application Priority Data
Sep. 22, 2000 (NZ) NZ507126

(51) Int. Cl. A61C 5/14; A61M 15/08

(52) U.S. Cl. 128/201.26; 128/207.18; 128/859; 606/234

(57) ABSTRACT

A system is disclosed for nasal delivery of Continuous Positive Airway Pressure to a user. The system includes a variable speed fan unit to pressurize the gases, and a humidification unit to provide a controllable level of humidity to the gases. The pressurized humidified gases are transported through a conduit to the user, and arc delivered through a nasal mask to the nasal passages. The oral cavity is sealed against “mouth leak” by insertion of a sealed mouthpiece. The mouthpiece includes an intra-oral sealing means and an extra-oral sealing means substantially sealing the oral cavity of said user. The extra-oral sealing means may be adjusted into one of two conditions, a first condition when said mouthpiece is inserted into said user's mouth being, substantially unengaged with said user's face, and a second condition when correctly positioned in said user's mouth being substantially engaged with said user's face and under compression thereupon.

### FIELD OF THE INVENTION

This invention relates to mouthpieces above ambient, and in particular to mouthpieces for use in conjunction with the nasal delivery of air in CPAP treatments of sleeping disorders such as sleep apnea

### BACKGROUND

Sleep apnea treatments have been significantly advanced with the introduction of continuous positive airway pressure (CPAP) treatments. These treatments, as introduced, involve the supply of gases from a gases supply or blower to a patient through a conduit and nasal mask to provide an elevated internal pressure in the users airways to assist the muscles to keep the airways open. This airstream is provided to the user through a nasal mask applied over the nose and held in place by a harness. This configuration has been almost universally adopted based on the well known observation that humans show a decided preference for nasal breathing dog sleep. For this reason there has been little development undertaken into other possible methods of providing the pressurized airstream to a user.

However in such cases of nasal masks it often evidenced that substantial leakage of the pressurised air can occur through the mouth of the user. For example, in U.S. Pat. No. 5560354 assigned to ResCare Limited, a mask is designed which includes two sealed compartments for both the nose and the mouth. While this is some ways overcomes the problems associated with nasal masks in preventing leakage from the mouth the apparatus described therein is somewhat bulky and uncomfortable for the user. Further since it is also attached to the nasal mask and is completely extra-oral it may still suffer from partial leakage around the side of the cushion.

### SUMMARY OF THE INVENTION

It is therefore an object of the present invention to provide a mouthpiece for nasal delivery of pressurized gases that goes some way toward overcoming the abovementioned disadvantages or will at least provide the public with a useful choice.

In a first aspect the present invention consists in a mouthpiece for nasal delivery of pressurised gases comprising:

a vestibular shield having an inner surface and an outer surface, said vestibular shield having a predetermined height which will overlap a user's teeth and gums when positioned in the mouth vestibule of a user;

extra-oral sealing means associated with said vestibular shield which may be adjusted into one of two configurations, a first condition when said mouthpiece is inserted into a user's mouth being substantially unengaged with a user's face, and a second condition when correctly positioned in a user's mouth being substantially engaged with a user's face and under compression thereupon.

Preferably, said extra-oral sealing means are detachable from said vestibular shield.

Preferably, said extra-oral sealing means are constructed of silicon rubber.

Preferably, said extra-oral sealing means comprise a tapared flap.

Preferably, said flap has a wide end and a narrow end said narrow end being attached to said vestibular shield.

Preferably, said first condition comprises said wide end being distal to a user relative to said narrow end being proximal to a user.

Preferably, said second condition comprises said wide end being proximal to a user relative to said narrow end being distal to a user.

Preferably, the compressive force between said vestibular shield and said extra-oral sealing means on the area surrounding a user's lips is sufficient to secure said mouthpiece in place on a user and to provide a substantial seal thereto.

Preferably, said wide end is adapted to conform to the facial contours of a user.

In a second aspect in a first aspect the present invention consists in a system capable of being used for oral delivery of gases pressurized above ambient to a user comprising;

gases supply means,

a gases passageway in fluid communication with said gases supply means,

a nasal mask in fluid communication with said gases passageway thereby supplying said gases to the nasal passages of said user, and

a mouthpiece including an intra-oral sealing means and an extra-oral sealing means substantially scaling the oral cavity of said user.

Preferably, said extra-oral sealing means may be adjusted into one of two conditions, a first condition when said mouthpiece is inserted into said user's mouth being substantially unengaged with said user's face, and a second condition when correctly positioned in said user's mouth being substantially engaged with said user's face and under compression thereupon.

Preferably, said intra-oral sealing means comprises a vestibular shield having an inner surface and an outer surface, said vestibular shield having a predetermined height which will overlap a user's teeth and gums when positioned in the mouth vestibule of a user;

Preferably, said extra-oral sealing means are detachable from said mouthpiece.

Preferably, said extra-oral sealing means are constructed of silicon rubber.

Preferably, said extra-oral sealing means comprise a tapered flap.

Preferably, said flap has a wide end and a narrow end, said narrow end being attached to said vestibular shield.

Preferably, said first condition comprises said wide end being distal to said user relative to said narrow end being proximal to said user.

Preferably, said second condition comprises said wide end being proximal to a user relative to said narrow end being distal to said user.

Preferably, the compressive force between said vestibular shield and said extra-oral sealing means on the area surrounding said user's lips is sufficient to secure said mouthpiece in place on said user and to provide a substantial seal thereto.

Preferably, said wide end is adapted to conform to the facial contours of said user.

Preferably, said system further comprises humidification mew disposed in the flow path at said gases between said gases supply means and said mouthpiece, adapted to humidify said gases to a desired level of humidity.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

The invention consists in the foregoing and also envisages constructions of which the following gives examples.

### BRIEF DESCRIPTION OF THE DRAWINGS

One preferred form of the present invention will now be described with reference to the accompanying drawings in which;

FIG. 1 is a side elevational view of the system according to the present invention as being used by a patient,

FIG. 2 is a perspective view of the mouthpiece with the outer flap in place,

FIG. 3 is a perspective view of the outer flap bent back, and

FIG. 4 is a cutaway view of the present invention with the outer flap in use.

### DETAILED DESCRIPTION OF THE PREFERRED EMBODIMENTS

While the invention may be susceptible to embodiment in different forms, there is shown in the drawings, and herein will be described in detail, specific embodiments with the understanding that the present disclosure is to be considered an exemplification of the principles of the invention, and is not intended to limit the invention to that as illustrated and described herein.

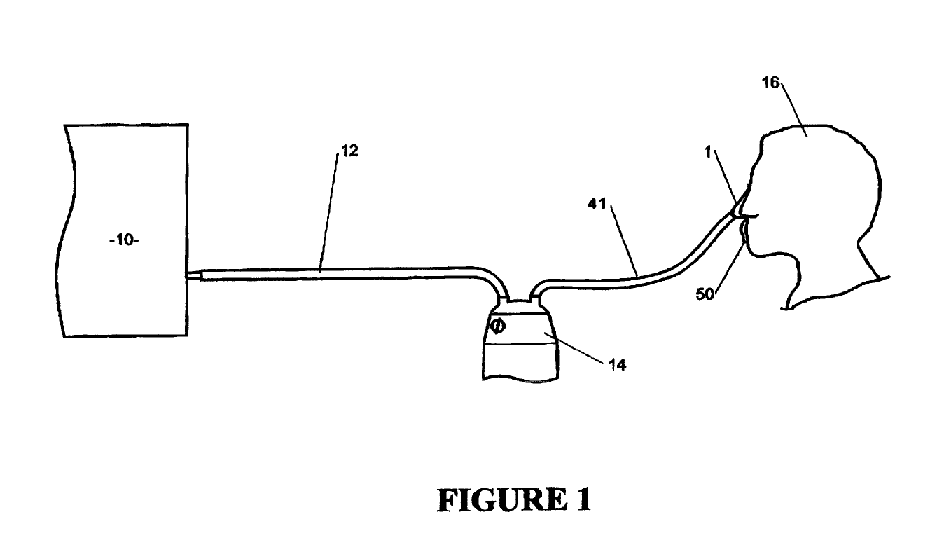

The present invention provides a novel system for nasal delivery of gases pressurised above ambient to a user and is especially suited for use in the nasal delivery of air in continuous positive airway pressure (CPAP) treatments of sleeping disorders such as sleep apnoea. As shown in FIG. 1, the system includes a respirator or blower 10, connected by conduit 12 to a humidifier 14. The humidified gases are delivered to the patient 16 through a nasal mask 1 which is connected to a breathing circuit 41, with a mouthpiece 50 sealing the oral cavity.

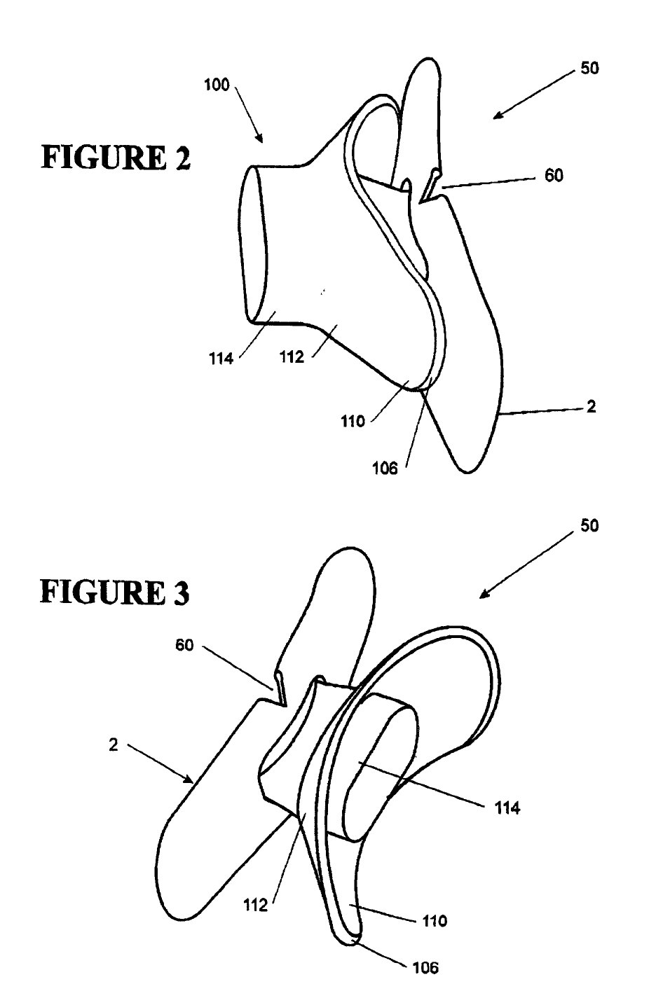

a preferred embodiment of the present invention is illustrated in FIG. 2 to 4. In this embodiment, the mouthpiece 50 includes a vestibular shield 2 being a generally flat and generally rectangularly-shaped member in front elevation having a curved profile that reflects the curvature of a user's jaw and in turn the curvature of the labial vestibule region. The major differences between the mouthpiece 50 and the embodiments described above arc provided on the inner face of the vestibular shield. In the mouthpiece 50 the sealing effect of the vestibular shield 2 against the lips of the user is enhanced by providing teeth abutments of significantly increased thickness than the raised area of the earlier embodiments. In particular, an upper teeth abutment 58 and a lower teeth abutment 59 are provided, with the lower teeth abutment 59 protruding further from the inner face of the vestibular shield 2 than the upper teeth abutment 58. This difference serves to match the typical over-bite of most users.

A notch 60 is provided centrally in the upper edge of the vestibular shield 2 to accommodate the upper frenal attachment. The vestibular shield 2 being relatively thin for additional suppleness and user comfort.

Referring particularly to FIG. 2, in its preferred form the mouthpiece 50 is preferably formed by over-moulding a soft and supple material part 70 over a stiffer material part 67. These can generally be termed the shield part and the forming insert. The forming insert preferably includes a par of upper and lower vertical flanges 63 and 64 to fully engage within the supple material. The upper and lower teeth abutments 58 and 59 are of the soft and supple material and are part of the over-moulding 70.

Referring now to FIGS. 2 and 3 of the present invention is illustrated with an extra-oral sealing flap 100 attached. The flap 100 in its natural bias (shown in FIG. 2) is tapered, the wide open end 110 of which is shaped to conform to the facial contours around the outside of the mouth of a user. The narrow end 112 joins to a cylindrical section 114, which is designed to slide over the stiff body 67 of the mouthpiece 50. While this is one method of attachment the flap 100 might also be constructed as an integral part of the over-moulding of the mouthpiece 50. The flap 100 needs to be constructed of flexible material, therefore materials such as silicone rubber can be employed to fashion the flap.

The outer flap 100 is seen in FIG. 3, in a bent back position. It will be appreciated that when the mouthpiece 50 is being inserted into the mouth of a user, the outer flap 100 is intended to be in this bent back position to aid insertion. Prior to insertion, the outer flap is bent back by simply pressing on its outer periphery 106, until it snaps into the bent back position, in which it will stay unaided.

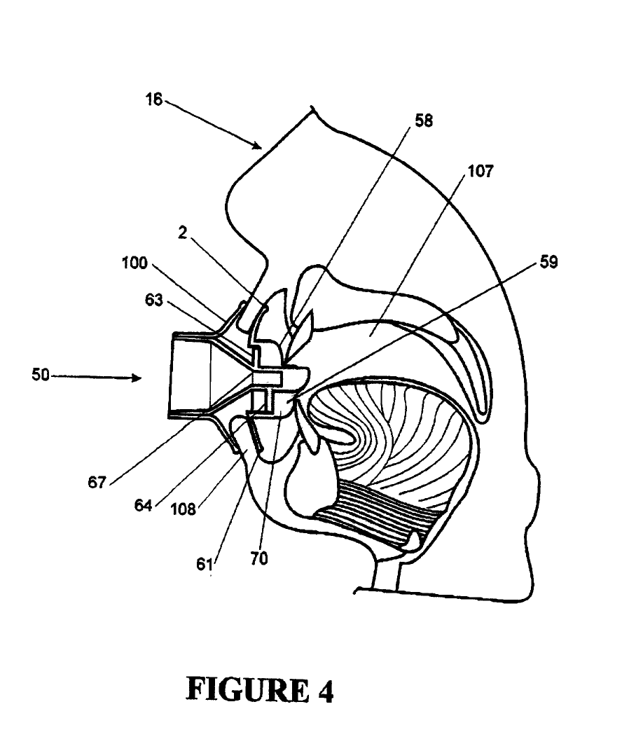

In FIG. 4 we see the outer flap 100 in use with the mouthpiece 50 in the mouth 107 of a user 16. Once correctly positioned in the mouth 107, the outer flap 100 may be adjusted into its operational position by pressing on its outer periphery 106 until it snaps back to press against the outside of the mouth 108. Due to the relative position of the vestibular shield 2 and the outer flap 100, the outer flap 100 is unable to fully reach its natural bias and thereby inflicts a compressive force on the outside of the mouth 108.

It will be appreciated that as well as providing a substantially airtight seal the addition of the outer flap provides enough compressive force on the mouth to keep the mouthpiece in place without the need for straps. This allows the administering of CPAP therapy to be considerably less obtrusive than traditional methods. Further it is can be seen that the present invention provides a system including mouthpiece 50 for nasal delivery of CPAP treatment which at once is low cost and effective. With the addition of the mouthpiece, an effective seal is created around the users mouth, avoiding any possibility of mouth leak, as night otherwise be encountered with nasal CPAP.

1. A mouthpiece for nasal delivery of pressurised gases comprising:
a vestibular shield having an inner surface and an outer surface, said vestibular shield having a predetermined height which will overlap a user's teeth and gums when positioned in the mouth vestibule of a user;
extra-oral sealing means associated with said vestibular shield which may be adjusted into one of two configurations, a first condition when said mouthpiece is inserted into a user's mouth being substantially unengaged with a user's face, and a second condition when correctly positioned in a user's mouth being substantially engaged with a user's face and under compression thereupon.

2. A mouthpiece as claimed in claim 1 wherein said extra-oral sealing means are adapted to be detachable from said vestibular shield.

3. A mouthpiece as claimed in claim 2 wherein said extras sealing means are constructed of silicon rubber.

4. A mouthpiece as claimed in claim 3 wherein said extra-oral sealing means comprise a tapered flap.

5. A mouthpiece as claimed in claim 4 wherein said flap has a wide end and a narrow end, said narrow end being attached to said vestibular shield.

6. A mouthpiece as claimed in claim 5 wherein said first condition comprises said wide end being distal to a user relative to said narrow end being proximal to a user.

7. A mouthpiece as claimed in claim 6 wherein said second condition comprises said wide end being proximal to a user relative to said narrow end being distal to a user.

8. A mouthpiece as claimed in claim 7 wherein the compressive force between said vestibular shield and said extra-oral sealing means on the area surrounding a user's lips is sufficient to secure said mouthpiece in place on a user and to provide a substantial seal thereto.

9. A mouthpiece as claimed in claim 8 wherein said wide end is adapted to conform to the facial contours of a user.

10. A system for oral delivery of gases pressurized above ambient to a user comprising;
gases supply means,
a gases passageway in fluid communication with said gases supply means,
a nasal mask in fluid communication with said gases passageway thereby supplying said gases to the nasal passages of said user, and
a mouthpiece including an intra-oral scaling means and an extra-oral sealing means substantially sealing the oral cavity of said user.

11. A system as claimed in claim 10 wherein said extra-oral sealing means may be adjusted into one of two conditions, a first condition when said mouthpiece is inserted into said user's mouth being substantially unengaged with said user's face, and a second condition when correctly positioned in said user's mouth being substantially engaged with said user's face and under compression thereupon.

12. A system as claimed in claim 11 wherein said intra-oral sealing means comprises a vestibular shield having an inner surface and an outer surface, said vestibular shield having a predetermined height which will overlap a user's teeth and gums when positioned in the mouth vestibule of a user.

13. A system as claimed in claim 12 wherein said extra-oral sealing means are detachable from said mouthpiece.

14. A system as claimed in claim 13 wherein said extra-oral sealing means are constructed of silicon rubber.

15. A system as claimed in claim 14 wherein said extra-oral sealing means comprise a tapered flap.

16. A system as claimed in claim 15 wherein said flap has a wide end and a narrow end, said narrow end being attached to said vestibular shield.

17. A system as claimed in claim 16 wherein said first condition comprises said wide end being distal to said user relative to said narrow end being proximal to said user.

18. A system as claimed in claim 17 wherein said second condition comprises said wide end being proximal to a user relative to said narrow end being distal to said user.

19. A system as claimed in claim 18 wherein the compressive force between said vestibular shield and said extra-oral sealing means on the area surrounding said user's lips is sufficient to secure said mouthpiece in place on said user and to provide a substantial seal thereto.

20. A system as claimed in claim 19 wherein said wide end is adapted to conform to the facial contours of said user.

21. A system as claimed in claim 20 further comprising humidification means disposed in the flow path of said gases between said gases supply means and said mouthpiece, adapted to humidify said gases to a desired level of humidity.

22. A method of oral delivery of gases pressurized above ambient to a user comprising the steps:
supplying said gases pressurized above ambient to the nasal passages of said user, and
substantially sealing the oral cavity of said user using a mouthpiece including an intra-oral sealing means and an extra-oral sealing means.
